(19) United States
(12) Patent Application Publication
(10) Pub. No.: US 2024/0398445 A1
(43) Pub. Date: Dec. 5, 2024

(54) IMPLANTS FOR GUIDED SPINAL GROWTH

(71) Applicant: OrthoPediatrics Corp., Warsaw, IN (US)

(72) Inventor: Richard Detlefsen, Warsaw, IN (US)

(73) Assignee: OrthoPediatrics Corp., Warsaw, IN (US)

(21) Appl. No.: 18/697,227

(22) PCT Filed: Sep. 30, 2022
(86) PCT No.: PCT/US2022/077379

§ 371 Date: Mar. 29, 2024

Related U.S. Application Data
(60) Provisional application No. 63/251,273, filed on Oct. 1, 2021.

(51) Int. Cl. A61B 17/70

(52) U.S. Cl. CPC A61B 17/7041; A61B 17/7034; A61B 17/7037

(57) ABSTRACT

One embodiment of an implantable anchor assembly for support of a spinal rod includes a bone connector; a head having first and second opposing arms and forming a lower rod pathway therebetween, each said first and second opposing arm having the same one of a notch or a projection, the one of a notch or projection of said first arm facing one of a notch or projection of said second arm; a rod retainer having third and fourth opposing arms and forming an upper rod pathway therebetween, each said third and fourth opposing arm having the other of a notch or a projection, said third and fourth opposing arms being slidable to a position between said first and second opposing arms, said rod retainer having a threaded aperture; and a set screw threadably receivable within the threaded aperture.

### CROSS REFERENCE TO RELATED APPLICATION(S)

This application claims the benefit of priority to U.S. Provisional Patent Application No. 63/251,273, filed Oct. 1, 2021, which is incorporated herein by reference.

### FIELD OF THE INVENTION

This invention describes various biocompatible implant, and including spinal implants with improved wear and low friction features.

### BRIEF DESCRIPTION OF THE DRAWINGS

Some of the figures shown herein may include dimensions. Further, the figures shown herein have been created from scaled drawings. It is understood that such dimensions, or the relative scaling within a figure, are by way of example, and not to be construed as limiting unless so stated in a claim. Persons of ordinary skill will also recognize that CAD renderings may include lines that pertain to changes in the computer model, and not necessarily to component features.

FIG. 1 is a front elevational view of an anchor assembly according to one embodiment of the present invention.

FIG. 2 is a side elevational view of the apparatus of FIG. 1.

FIG. 3 is a front, top, perspective, cross sectional view of the apparatus of FIG. 1.

FIG. 4 is a front elevational, cross sectional view of the apparatus of FIG. 1.

FIG. 5 is a top plan view of the apparatus of FIG. 1.

FIG. 6A is a top plan view of a partial assembly of the apparatus of FIG. 1.

FIG. 6B is a side elevational and orthogonal view of the apparatus of FIG. 6A.

FIG. 6C is a side elevational and orthogonal view of the apparatus of FIG. 6B.

FIG. 7A is a top plan view of a portion of the apparatus of FIG. 1.

FIG. 7B is a side elevational and orthogonal view of the apparatus of FIG. 7A.

FIG. 7C is a side elevational and orthogonal view of the apparatus of FIG. 7B.

FIG. 7D is a side, top, perspective view of the apparatus of FIG. 7A.

FIG. 8A is a top plan view of yet another portion of the apparatus of FIG. 1.

FIG. 8B is a side elevational and orthogonal view of the apparatus of FIG. 8A.

FIG. 8C is a side elevational and orthogonal view of the apparatus of FIG. 8B.

FIG. 8D is a side, top, perspective view of the apparatus of FIG. 8A.

FIG. 9A is a side elevational, cross sectional view of a portion of the assembly of FIG. 1.

FIG. 9B is a side elevational, cross sectional view of a portion of the assembly of FIG. 9A, and presented orthogonally to FIG. 9A.

FIG. 10A is a side elevational, cross sectional, exploded view of the apparatus of FIG. 6A.

FIG. 10B is a side, top, perspective, semi-transparent view of a portion of the apparatus of FIG. 1.

FIG. 11 is a front elevational view of an implantable anchor assembly according to another embodiment of the present invention.

FIG. 12 is a cross sectional view of the apparatus of FIG. 11.

FIG. 13 shows the apparatus of FIG. 12 with the head rotated relative to the bone connector.

FIG. 14 is a side, top, perspective and exploded view of the apparatus of FIG. 11.

FIG. 15 is a side elevational view of the apparatus of FIG. 14.

FIG. 16 shows a pair of the implantable apparatus of FIG. 11, shown implanted in a vertebrae, and coupled to a rod.

FIG. 17 is a front, top, perspective view of a portion of an implantable anchor assembly according to another embodiment of the present invention, and shown with a locking tool.

FIG. 18 is a cross sectional view of the apparatus of FIG. 17.

FIG. 19 is a view of the apparatus of FIG. 18 with the bone connector and head interlocked to each other.

FIG. 20 is an exploded view of an implantable anchor assembly according to another embodiment of the present invention.

FIG. 21 is a side elevational, cross sectional, exploded view of the apparatus of FIG. 20.

FIG. 22 is a side elevational view of the assembled apparatus shown in FIG. 20.

### ELEMENT NUMBERING

The following is a list of element numbers used with all of the embodiments, and at least one noun used to describe that element. The “X” for all of these numbers is removed or replaced with a number (0 or greater) in the text and drawings of this application. Consistent with statements made elsewhere in this specification, these various 2-digit element numbers are used among multiple embodiments, and aspects of a particular element stated for one embodiment can be applied to the same element number in a different embodiment, except as shown and described differently, and as would be understood by a person of ordinary skill in the art. It is understood that none of the embodiments disclosed herein are limited to these nouns, and these element numbers can further include other words that would be understood by a person of ordinary skill reading and reviewing this disclosure in its entirety.

| | | | | | |
|---|---|---|---|---|---|
| 10 | bone | f | centerline | a1 | engagement |
| X20 | implantable | g | screw head | | feature; notch |
| | anchor assy. | h | corridor | | or projection |
| X24 | rod pathway | X29 | bone connector | b1 | interlocking |
| a | entrance/exist | | coupling | | feature; |
| b | central passage | a | ring | | dovetail |
| c | min. cross | b | groove | c | threaded |
| | sectional | c | flexible | | portion |
| | distance | | coupling wire | d | top surface |
| f | aperture | X30 | head for | e | facing portion |
| 28 | bone connector | | supporting a | f | centerline |
| | assembly | | sliding rod | X34 | lower rod |
| a | captured end | a | body | | pathway |
| b | curved outer | b | side | a | entrance/exit |
| | surface; | c | rod surface | b | lower central |
| | rounded head | d | central opening | | passage |
| c | threaded shaft | e1 | corner | c | minimum cross |
| d | cannula | e2 | corner | | sectional |
| e | tool coupling | X31 | arm | | distance |
| | feature | g | channel | d | notch or |
| X35 | through | h | direction of | | projection |
| | passageway | | compression | b4 | necked-down |
| f | centerline | X52 | flexible portion | | portion |
| g | threaded | X54 | upper rod | c | through |
| | portion | | pathway | | passage |
| | engages set | a | entrance/exit | X64 | upper rod |
| | screw | b | upper central | | pathway |
| h | seat for set | | passage | a | entrance/exit |
| | screw | c | minimum cross | b | upper central |
| X37 | pocket for bone | | sectional | | passage |
| | connector | | distance | X66 | channel for rod |
| | assembly | d | notch or | | retainer |
| f | axis of rod | | projection | X80 | set screw |
| | pathway center | f | living hinge; | X81 | head |
| | and connector | | notch; | X82 | overextending |
| | center | | weakened | | lip |
| g | included angle | | portion | X85 | threads |
| X39 | tool grasping | X55 | threaded hole | X86 | locking member |
| | features | X56 | channel for | a | pin; projection |
| X50 | rod retainer | | screw | b | grip; holding |
| a | body | a | corner | | portion |
| b | side | X58 | projection | X88 | aperture |
| X51 | arm; end wall | X59 | lower rod | X89 | tool coupling |
| a1 | engagement | | contact hoop | | feature |
| | feature; notch | X60 | cap | X90 | rod |
| | or projection | a | body | X91f | centerline |
| a2 | weakened | b | side | | |
| | portion; | X61 | arm | | |
| | keyhole; notch | b1 | interlocking | | |
| b1 | interlocking | | feature; | | |
| | feature; | | dovetail | | |
| | dovetail | b3 | outermost | | |
| b2 | elastically | | portion | | |
| | bendable | | | | |
| | portion | | | | |

### DETAILED DESCRIPTION OF ONE OR MORE EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates. At least one embodiment of the present invention will be described and shown, and this application may show and/or describe other embodiments of the present invention, and further permits the reasonable and logical inference of still other embodiments as would be understood by persons of ordinary skill in the art.

It is understood that any reference to “the invention” is a reference to an embodiment of a family of inventions, with no single embodiment including an apparatus, process, or composition that should be included in all embodiments, unless otherwise stated. Further, although there may be discussion with regards to “advantages” provided by some embodiments of the present invention, it is understood that yet other embodiments may not include those same advantages, or may include yet different advantages. Any advantages described herein are not to be construed as limiting to any of the claims. The usage of words indicating preference, such as “various embodiments” or “preferably,” refers to features and aspects that are present in at least one embodiment, but which are optional for some embodiments, it therefore being understood that use of the word “preferably” implies the term “optional.”.

The use of an X-series prefix for an element number (XYY.YY) refers to an element that is the same as the non-prefixed element (YY.YY), except as shown and described. As an example, an element 1020.1 would be the same as element 20.1, except for those different features of element 1020.1 shown and described. Further, common elements and common features of related elements may be drawn in the same manner in different figures, and/or use the same symbology in different figures. As such, it is not necessary to describe the features of 1020.1 and 20.1 that are the same, since these common features are apparent to a person of ordinary skill in the related field of technology. Further, it is understood that some features 1020.1 and 20.1 may be backward compatible, such that a feature of a later discussed embodiment (XNYY.YY) may include features compatible with other various embodiments that were discussed earlier (MYY.YY), as would be understood by those of ordinary skill in the art. This description convention also applies to the use of prime (′), double prime (″), triple prime (′″) and star or asterisk (*) suffixed element numbers. Therefore, it is not necessary to describe the features of 20.1, 20.1′, 20.1″, 20.1″ and 20* that are the same, since these common features are apparent to persons of ordinary skill in the related field of technology.

Although various specific quantities (spatial dimensions, temperatures, pressures, times, force, resistance, current, voltage, concentrations, wavelengths, frequencies, heat transfer coefficients, dimensionless parameters, etc.) may be stated herein, such specific quantities are presented as examples only, and further, unless otherwise explicitly noted, are approximate values, and should be considered as if the word “about” prefaced each quantity. Further, with discussion pertaining to a specific composition of matter, that description is by example only, and does not limit the applicability of other species of that composition, nor does it limit the applicability of other compositions unrelated to the cited composition.

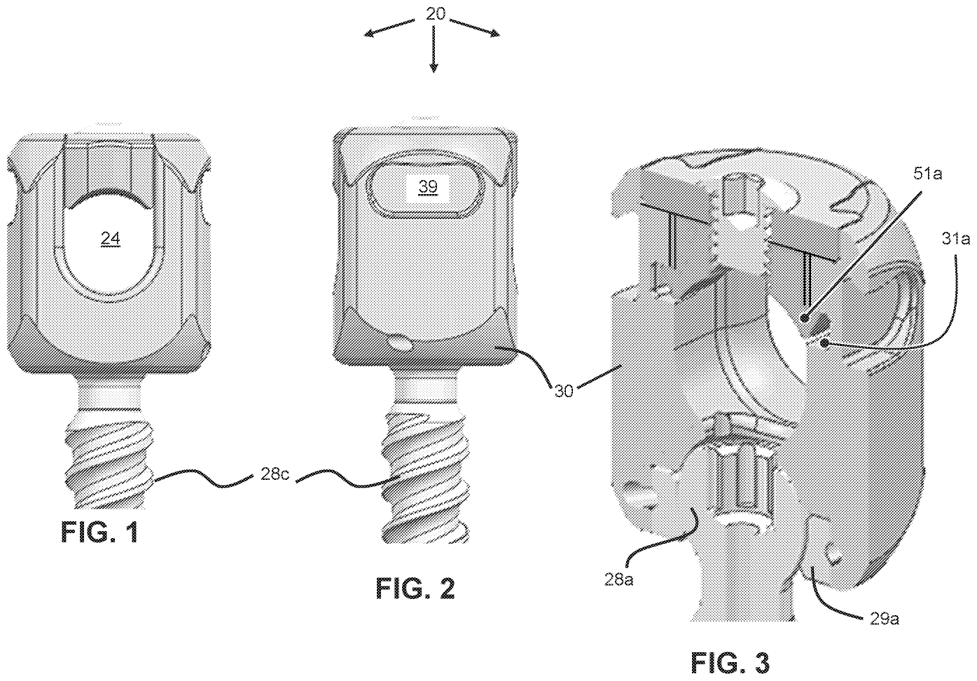

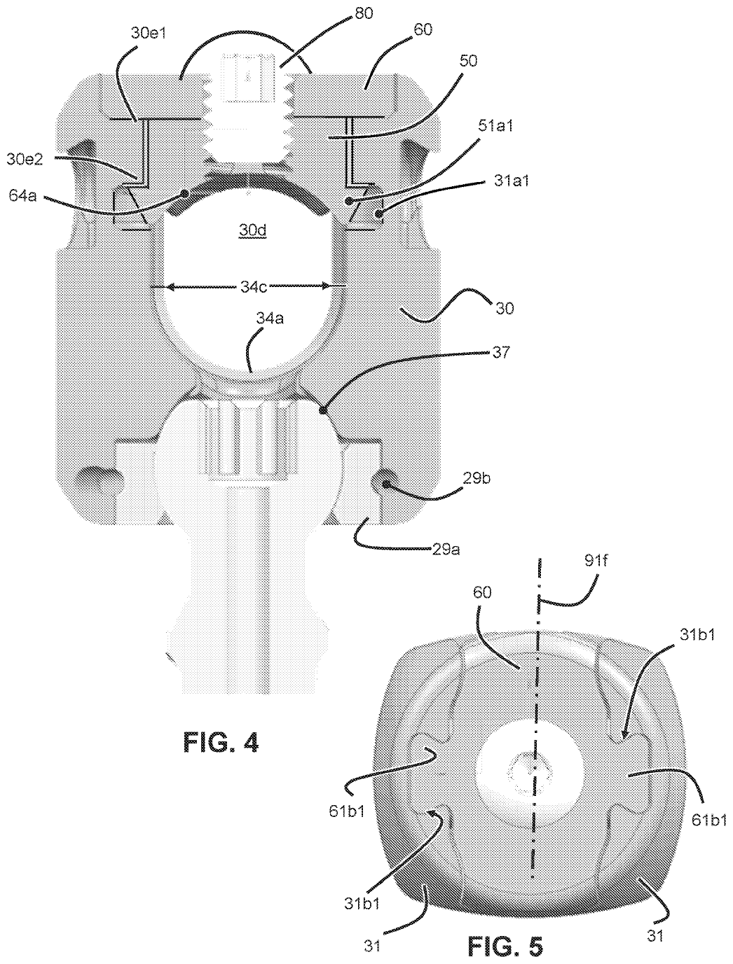

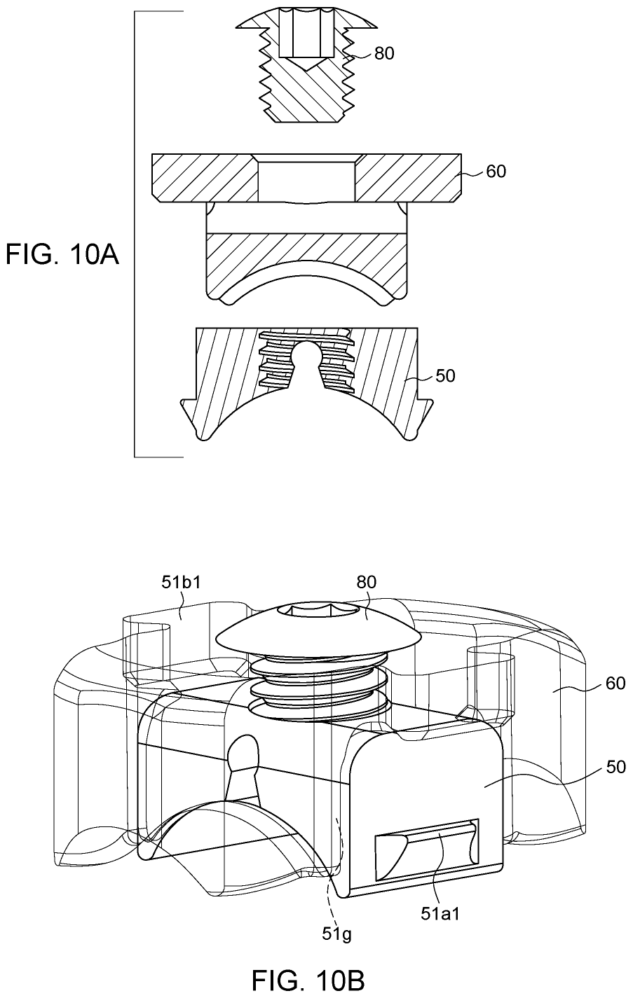

FIGS. 1-10 present various views of an implantable spinal anchor assembly 20 according to one embodiment of the present invention. Anchor assembly 20 includes a head 30 adapted and configured for sliding support of a rod (not shown), AND preferably adapted for polyaxial support by a bone connector assembly 28, such as the bone screw shown. As best seen in FIG. 4, the rounded head 28b is captured to head 30 by a connector coupling ring 29a in conjunction with a flexible coupling wire 29c that resides within a groove 29b. As shown, head 30 is free to rotate in three orthogonal dimensions relative to connector assembly 28.

It can be seen that bone connector assembly 28 preferably includes an end 28a with a curved outer surface 28b that is captured or contained within a pocket 37 of head 30. In some embodiments, this connection between the bone connector and the head provides polyaxial movement of the head 30 relative to the captured end 28a. Although what is shown and described are head to bone connector attachments that provide three degrees of relative rotational motion, other embodiment of the present invention contemplate connector to head couplings with two degrees of relative rotational motion, or one degree of relative rotational motion, or rigid attachment. Further, although a bone screw is shown and described having a threaded shaft 28c, yet other embodiments of the present invention contemplate any manner of coupling head 30 to a bone, including as examples, spikes that penetrate into the bone and flexible bands that couple the head to the bone.

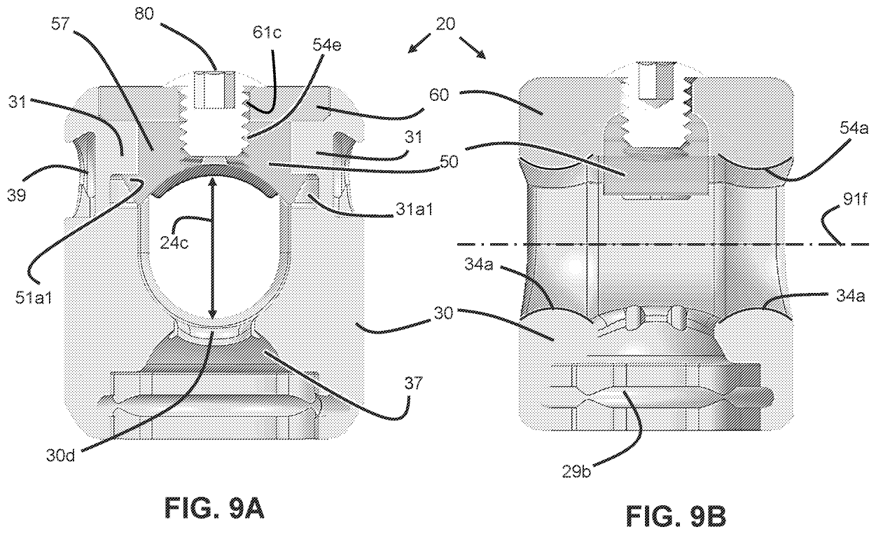

In some embodiments, head 30 is sometimes referred to as a tulip head, having two opposing arms 31 on either side of a central opening 30d. Opening 30d is adapted and configured to permit installation of a rod from the top of the central opening onto a lower rod pathway 34. Rod pathway 34 preferably includes a rounded inlet and rounded inlets/outlets 34a, as shown in FIG. 9B. In some embodiments, the central portion of the lower rod pathway expands to a diameter that is larger than the diameter of the entrance or exit, all preferably centered about a centerline 91f that generally defines a pathway for a rod (not shown).

It is understood that the entrance and exit to both the upper and lower rod pathways preferably include rounded entrances and exits similar to that shown in FIG. 9B, and which expand to a larger central region of the rod pathway. In some embodiments, the rod pathway 24 (which is the combination of the upper and lower rod pathways 54 and 34, respectively) is adapted and configured to permit freely sliding movement of a rod, and still further to accommodate rods that are bent.

Preferably, the minimum cross sectional distance 34c of lower rod pathway 34 is larger than the outer diameter of the associated rod, and still further that the minimum cross sectional distance 24c of the installed upper assembly is greater than the outer diameter of the rod. By selection of pathway dimensions that are greater than the outer dimensions of the rod (whether the rod has a circular cross section or a non-circular cross section), sliding motion of the rod relative to the head is provided, and frictional capture of the rod within the head is minimized.

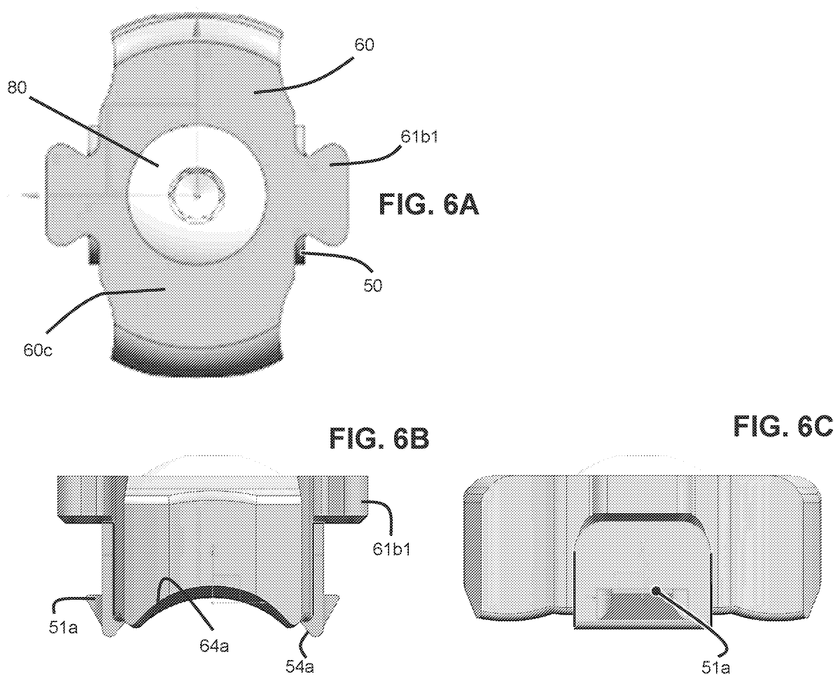

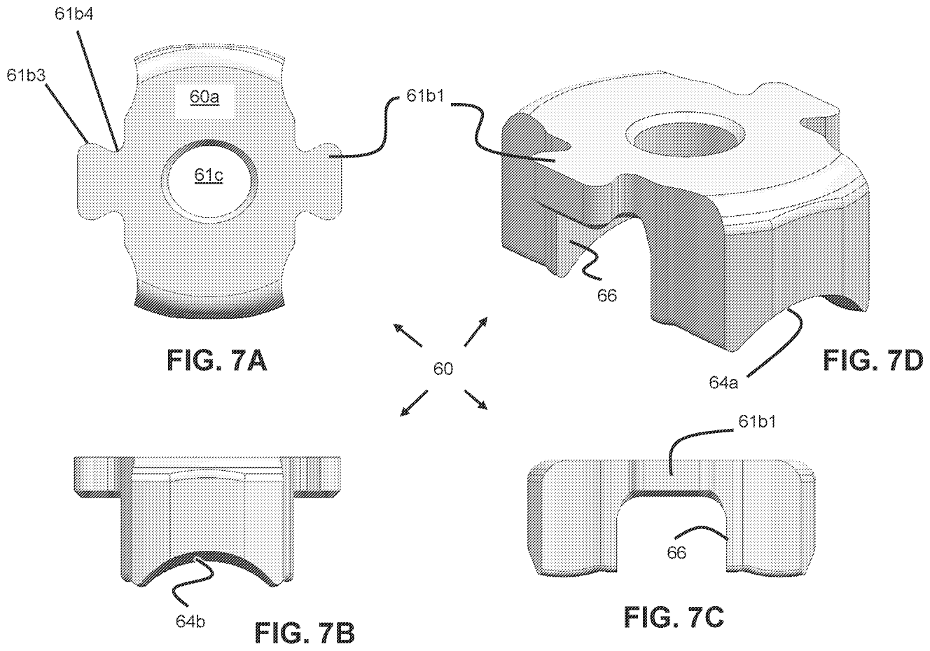

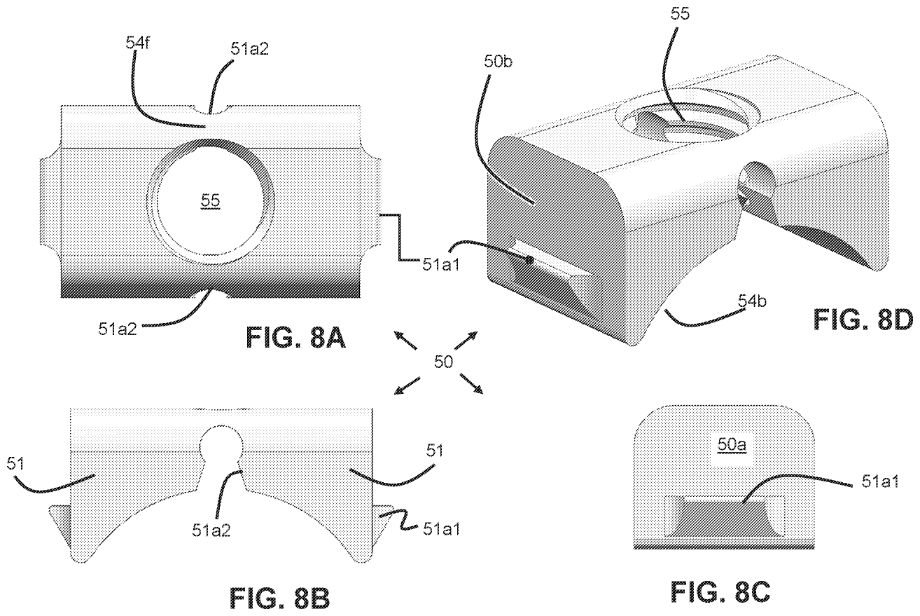

The body 30a of head 30 is adapted and configured with features that accept the combination of rod retainer 50 and cap 60, which are shown assembled in FIG. 6. Cap 60 is shown by itself in FIGS. 7 and rod retainer 50 is shown by itself in FIG. 8.

Referring to FIG. 7A and FIG. 5, it can be seen that the top surface is adapted and configured with an interlocking feature 31b1 that is adapted and configured to fit with a corresponding, preferably complementary-shaped feature 61b1 of cap 60. As shown in this embodiment, feature 61b is generally that of a male dovetail shape, and feature 31b1 is preferably a female counterpart to that dovetail shape. As best seen in FIG. 5, in some embodiments the cap 60 can be slid into the central opening 30d from above (i.e., into the plane of FIG. 5).

Once inserted with features 31b1 and 61b1 interlocked, it is to be noted that the cap and head are adapted and configured to prevent relative lateral sliding motion in two directions (i.e., within the plane of FIG. 5). Relative sliding motion along axis 91f (referring to FIG. 5) is prevented by the mechanical interference of the male and female dovetail features in that direction. Still further, in a direction perpendicular to axis 91f (i.e., sideways in FIG. 5) the interlocking of the dovetails further prevents any relative separation of the left and right arms 31 from one another. It is noted that the interlocking feature 61b1 of cap 60 (referring to FIG. 7) includes a wider most portion 61b3 that is coupled to body 60a by a narrower, necked-down portion 61b4. When interlocking feature 61b1 is placed within the corresponding interlocking feature 31b1, the co-action of the narrower attachment section with the wider, outermost section provides mechanical interference that prevents any relative separation or splaying apart of left and right arms 31.

Although what has been described is a cap having a male dovetail that fits within a correspondingly shaped female dovetail, it is further understood that the present invention contemplates embodiments in which the cap includes the female dovetail feature, and the head includes correspondingly shaped male dovetail feature. Still further, it is to be noted that the interlocking of the arms 31 of head 30 by cap 60 can be accomplished with a variety of different shapes other than a dovetail shape. Preferably, and more generally, the relative shapes of the cap interlocking features with the head interlocking features are adapted and configured to prevent lateral sliding of the cap relative to the head (i.e., in the two orthogonal dimensions in the plane of FIG. 5), yet permit sliding motion of the cap onto the head (i.e., perpendicular to the plane of FIG. 5).

FIG. 8 present various aspects of a rod retainer 50 according to one embodiment of the present invention. Rod retainer 50 preferably includes a body 50a having opposing sides 50b and a threaded aperture 54e in-between sides 50b. The underneath (rod-racing) bottom side of retainer 50 includes a concave, curving section 54b that constitutes the upper rod pathway central passage. Preferably, the midsection of body 50a includes a weakened portion 51a2 that creates a living hinge 54f on either side of threaded hole 55. The hinge section 54f coacts with engagement features 51a1 (preferably, one such feature on each side) to provide a means for elastically engaging rod retainer 50 with head 30.

Referring to FIG. 4, it can be seen that rod retainer 50 is slidingly inserted vertically downward into central opening 30d. As retainer 50 is vertically inserted (referring to FIG. 4, top to bottom) into central opening 30d, it can be seen that the outward and angled face of engagement feature 51a1 will contact a top corner 30e1 on each arm 31. After this top corner contacts the angled face of engagement feature 51a1, further vertical movement results in the corners 30e1 pressing against the angled faces. However, because of the living hinges 54f, the opposing sides 50b and their corresponding engagement features 51a1 are able to bend inward (i.e., toward the vertical centerline of the head 30), and thereby move out of the way of corner 30e1 and permit continued downward sliding.

Once the angled faces of feature 51a1 slide downward past a second corner 30e2, the engagement features 51a1 are able to elastically snap outwardly (towards their initial state) into a notch or channel 31a1 located in each arm 31. As features 51a1 preferably elastically snap back to their initial positions, a top face of each engagement feature moves into a position in which it mechanically interferes with the top of notch 31a1, thus preventing pullout of retainer 50 from head 30. In order to assure that engagement features 51a1 do not come out of engagement with corresponding engagement features 31a1, the insertion of set screw 80 within threaded hole 55 restores sufficient stiffness to the midsection of rod retainer 50 that any bending of arms 51 (that was previously permitted by the living hinges 54f) is now prevented because of the increased stiffness provided by the inserted set screw.

Although what has been shown and described are a pair of engagement features comprising a projection 51a1 and a notch 31a1, it is understood that the projection can also be located on head 30, and the notch or relieved feature 31a1 can be located on the rod retainer. Still further, yet other embodiments of the present invention contemplate engagement features of the rod retainer and head in a variety of different shapes. Further, although rod retainer 50 includes a living hinge 54f located in the central portion of body 50a, a living hinge-type portion can be located locally to feature 51a1, such as on the surface 50b. Further, the engagement features (generically, of a notch and a projection) can include springs (such as coil springs) to facilitate the elastic movement of the engagement feature as the rod retainer slides into place.

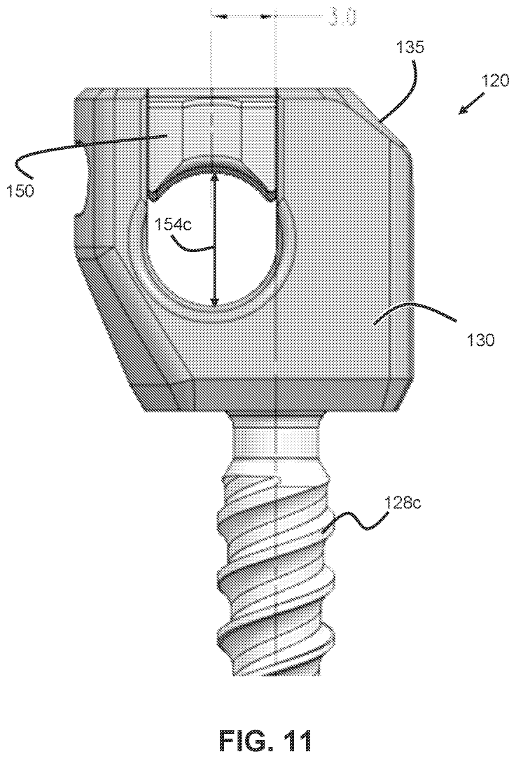

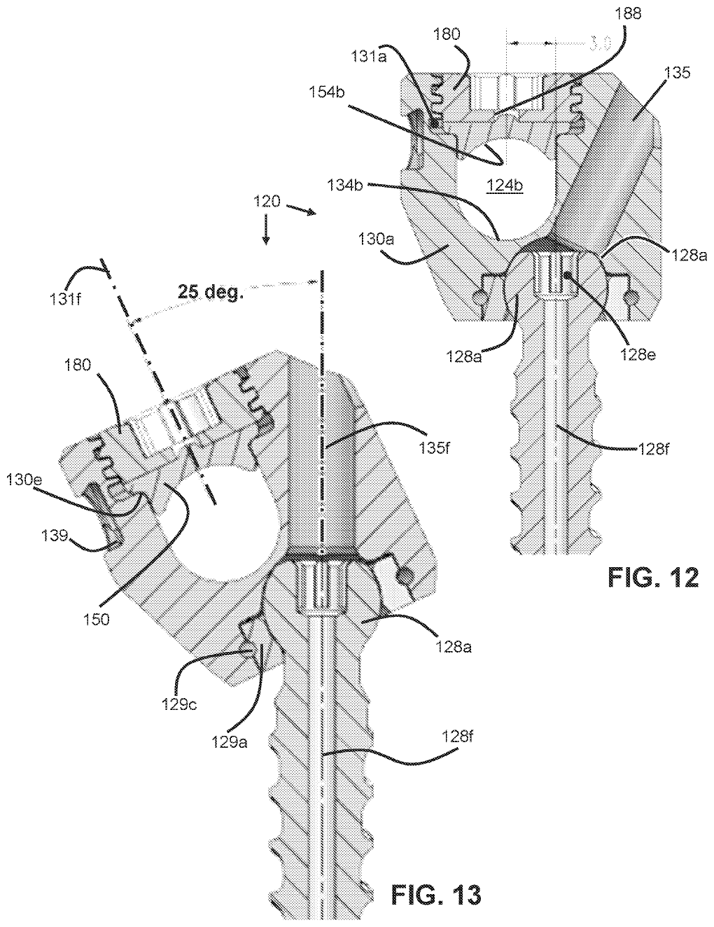

FIGS. 11-16 show various aspects of an implantable anchor assembly 120 according to one embodiment of the present invention. Anchor assembly 120 includes a head 130 that is preferably adapted and configured for polyaxial motion of the head relative 130 relative to the bone connector 128. In some respects, the coupling of anchor head 130 to the rounded head 128b is similar to that described for anchor assembly 20. However, as best seen in comparing FIGS. 12 and 13, anchor 120 provides a different range of rotation of head 130 relative to connector 128, as best seen in comparing FIGS. 12 and 13.

Anchor assembly 120 includes a head 130 having a central rod pathway 124b that is laterally offset from the preferably partially spherical pocket 137 that houses end 128 of the bone connector, as can be seen in FIG. 12. Referring to FIG. 13, it can be seen that because of this lateral offset that there is sufficient clearance of pathway 124 that a through passageway 135 can be placed in body 130a, with this passageway 135 opening into the connector pocket 137. Head 130 can be tilted sufficiently relative to connector centerline 128f, such that centerline 128f can become coaxial with centerline 135f of passageway 135.

The connector assembly 120 of FIG. 13 shows, as one example, an angular offset from passageway centerline 135 and the centerline 131f of the threads 131c of the arms 131. As shown in FIG. 13, the surgeon can insert a tool through passageway 135 and engage tool coupling feature 128e located at the captured end 128a. After the bone connector assembly has been tightened, head 130 can be rotated within the range of angles permitted by connector coupling assembly 129, and within the range of angles as is useful to the surgeon.

In comparing FIG. 13 with FIG. 3, note that the hole to provide access to the head of the bone connection screw is provided by aperture 30d in anchor 20, but through aperture 135 in anchor 120. By laterally offsetting the central rod path 124b from the pocket 137 and also providing access through a hole 135 separate from the rod path, anchor 120 includes a lower rod pathway 134 that is smooth and continuous preferably along its length, in comparison of the surface of lower rod pathway 34 which is interrupted by aperture 30d. By having a smooth and continuous surface uninterrupted by any apertures, a lesser degree of rod wear or headwear will be realized as the rod slides within the rod pathway 124. It has been found that interrupted rod surfaces (such as that shown in FIG. 9A) cause wear by the sliding of the rod outer surface against the edges of the connector access aperture, even if the edges of the aperture are rounded. In contrast, head 130 provides a lower rod pathway that is smooth and continuous from entrance to exit, preferably with no surface interruptions.

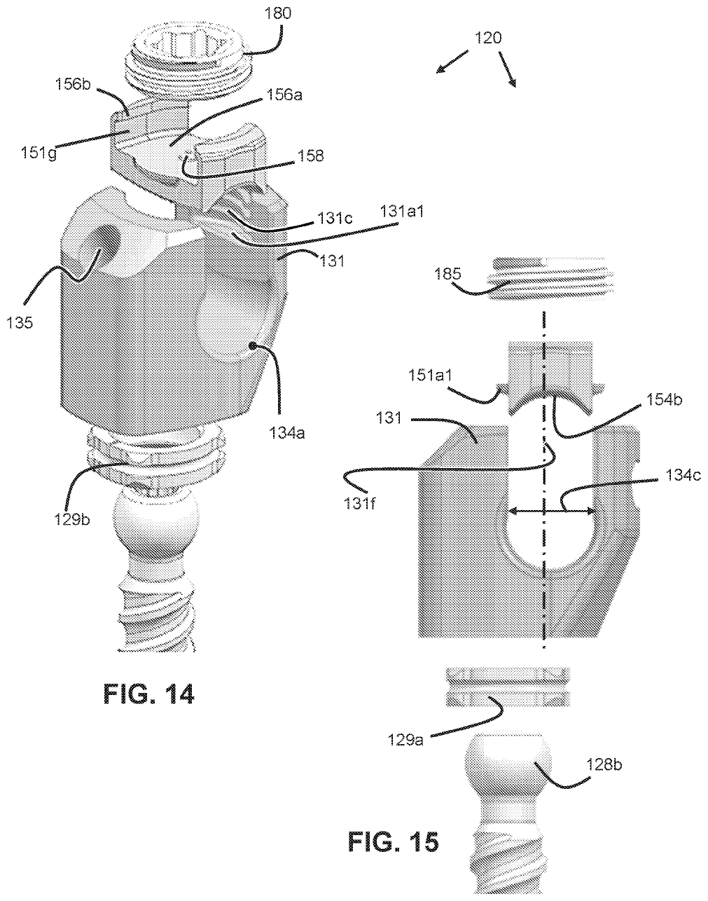

FIGS. 14 and 15 illustrate exploded implant assemblies 120. In the embodiment shown, a set screw 180 is located within a channel 151g of retainer 150. In some embodiments, screw 180 includes an aperture 188 that coacts with a projection 158 to provide an axis of rotation for tightening of set screw 180 within the threaded portions 131c of each of the facing and opposing arms 131. Preferably, channel 151g includes a top overextending lip 156b, with the major diameter of the set screw threads residing between the lip 156b and the bottom surface 156a. In this manner, set screw 180 is loosely captured within rod retainer 150.

Referring to FIG. 15, it can be seen that the opposing sides of retainer 150 each include an engagement feature 151a1 that projects outwardly, and which are adapted and configured to rest on top of a ledge 131a1 located immediately beneath threads 131c. Referring to FIGS. 12 and 13, it can be seen that after set screw 180 is fully tightened within the threads of arms 131, that each engagement feature 151a1 resides on a corner 130e. Tightening of the set screw compresses engagement feature 131a1 between the bottom of the set screw and the corner 130e. After the set screw is tightened, the top and bottom rod pathways combine to form the overall rod pathway 124. Note that in some embodiments, rod retainer 150 does not include a living hinge or weakened section that otherwise permit bending of retainer 150.

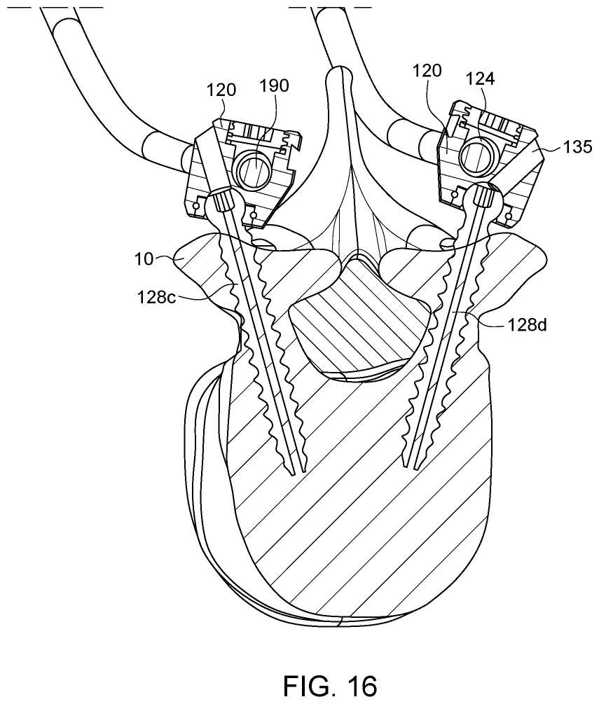

FIG. 16 shows a pair of anchor assemblies 120 that have been inserted into a vertebrae 10. The assembly 120 on the left of FIG. 16 shows a head 130 that has been tilted to provide access from the top to the bone connector 128. The anchor assembly 120 shown to the right of FIG. 16 is pivoted to a position in which passageway 135 does not permit tightening of the head of the bone screw. In both anchor assemblies 120, it can be seen that the rod pathway 124 provides clearance to rod 190, such that rod 190 can slide along its length relative to the anchor.

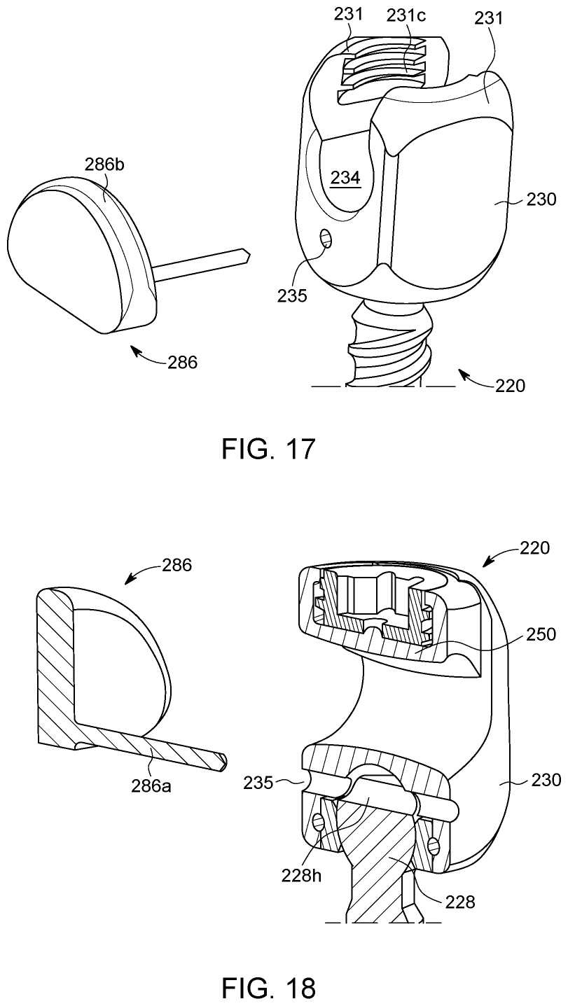

FIGS. 17-19 show various aspects of an implantable anchor assembly 220 according to another embodiment of the present invention. Anchor assembly 220 includes a head 230 that is adapted and configured for polyaxial support by a bone connector 228 in a manner similar to that previously shown, but with modifications that permit a temporary locking of the orientation of the head relative to the bone connector.

Head 230 includes a pair of facing, opposing arms 231, each of which includes a threaded section 231c. Situated between the arms 231 is an open channel having a lower rod pathway 234, and between which a rod can be vertically inserted. After the rod is inserted, the open channel can be closed by a rod retainer 250, to which is rotatably coupled to a set screw 280.

Implant 220 includes an aperture 235 that extends laterally across the internal pocket 237 that receives the end 228a of connector 228. Preferably, through passageway 235 extends completely across the lower portion of head 230, such that apertures are present on opposing sides of pocket 237. In addition, the end 228a of connector 228 preferably includes a corridor 28h that is located so that the corridor can be aligned with passageways 235 to form a single passageway that extends across the body 230a of head 230.

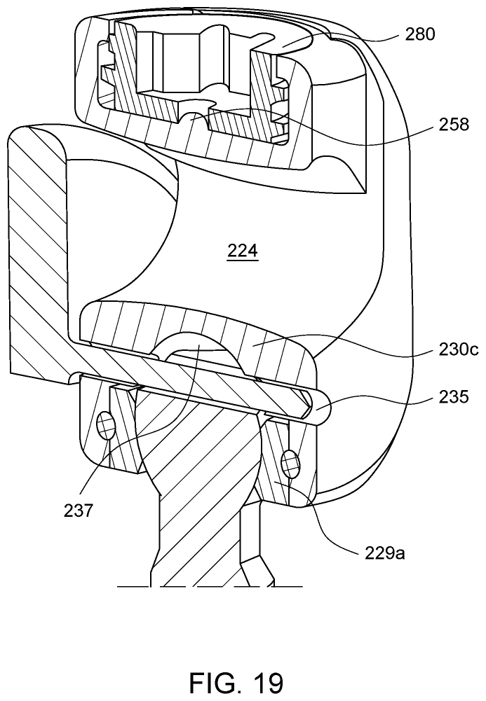

The embodiment of FIGS. 17-19 further includes a separable tool 286 that can be used to temporarily lock the relative positions of head 230 at connector 228. As best seen in FIG. 19, the projection 286a can be placed within the aligned corridor 228h and passageways 235. When this pin or projection 286a is located as shown in FIG. 19, the surgeon can apply a torque to head 230, with that torque being applied by pin 286 to connector 228. In this manner, the bottom surface of lower rod pathway 234 does not need to be interrupted for access to the bone connector head, and can be substantially smooth and continuous in areas that could be contacted by a rod. Again referring to FIG. 19, it can be seen that there is no vertical access through head 230 and into the pocket 237. Therefore, lower rod pathway 234 is substantially uninterrupted with holes or other features that would otherwise act as stress risers during sliding contact, with the possible generation of loose pieces worn off the rod.

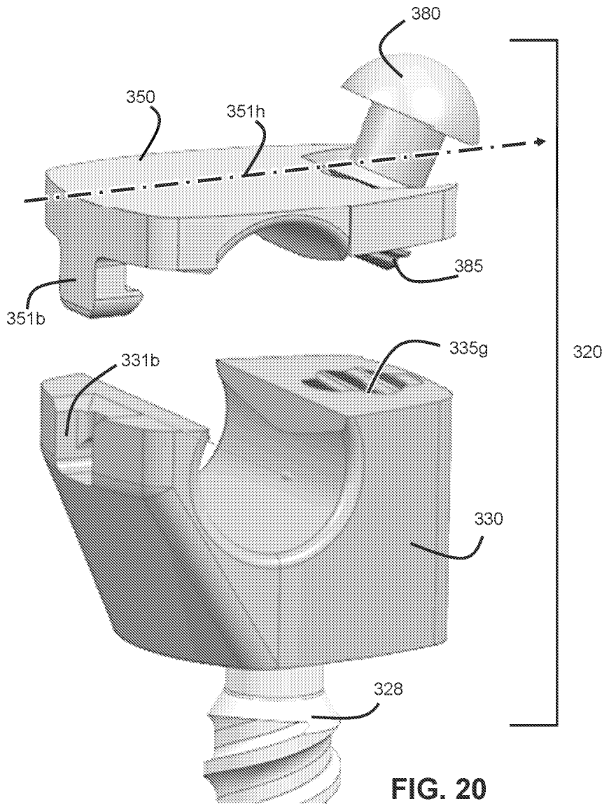

FIGS. 20-22 show various aspects of an implantable anchor assembly 320 according to another embodiment of the present invention. Anchor 320 includes a head 330 that is adapted and configured for polyaxial support by a bone connector 328. Preferably, the interconnection of rounded head 328 within pocket 337, such as by a coupling assembly 329, is similar to that described for anchor assembly X20.

Anchor assembly 320 includes a rod retainer 350 having an engagement feature 351b that interlocks with a corresponding head engagement feature 331b1, in coaction with a set screw 380 that is received within a through passage 335 that extends from the exterior of head 330 and into pocket 337. Anchor 320 includes a rod pathway 324 that is laterally offset and angularly from connector pocket 337, in a manner similar to that shown for anchor 220. Further, it can be seen that the center of rod pathway 324 and the center of connector pocket 337 establish an axis 337f, and that the included angle 337g between axis 337f and 335f is adapted and configured such that the major diameter of the threads 335g do not break into the smooth and continuous surface of rod pathway 324.

Preferably, angle 335g is greater than about twenty-five degrees, although the invention further contemplates any angular or linear separation of bore 335 and pathway 324 such that the internal, rod-sliding surface of pathway 324 remains uninterrupted by any aperture or any non-smooth and non-continuous feature. Still further, in a manner similar to that of implant 120, head 330 may be rotated relative to end 328 such that a tool placed within bore 335 can provide a tightening torque to end 328, without any need for a through hole within lower rod pathway 334 (which otherwise would also provide access to end 328a).

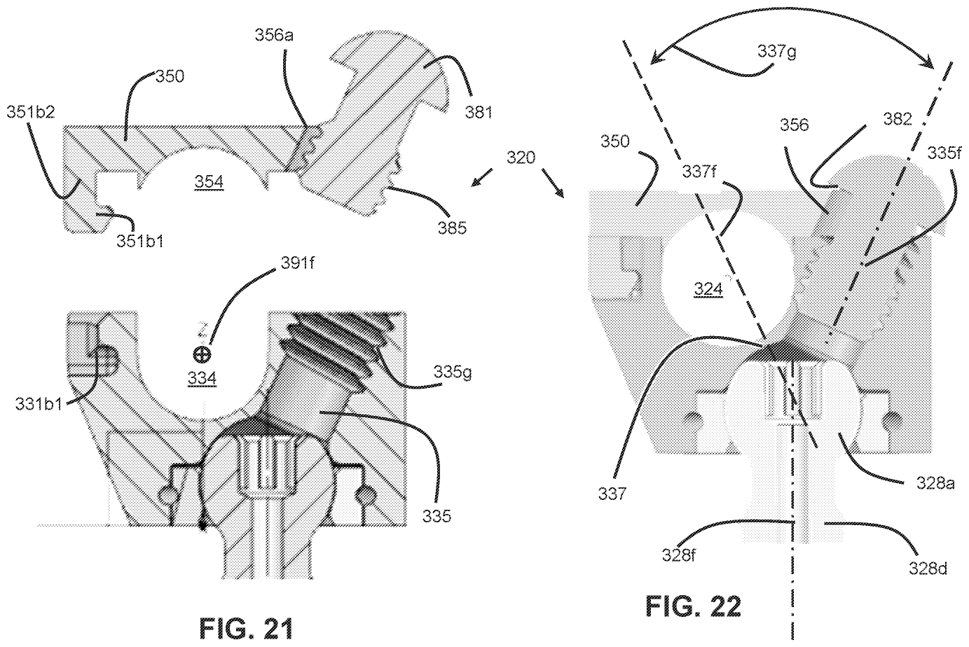

As shown in FIG. 20 engagement features 351b and 331b coact as a hook feature received within a recess feature, respectively. This engagement is shown in FIG. 22. In FIG. 22, it can be seen that engagement feature 351b readily slides into engagement with feature 331b along a lateral, side to side direction (right to left in the plane of FIG. 22). However, the hooking or coupling of engagement features 351b and 331b prevent any vertical pullout or separation of retainer 350 from head 330 (vertical, referring to bottom to top in the plane of FIG. 22). Referring to FIG. 20, it can be seen that the engagement of features 351b and 331b are further prevented from side to side disengagement because of mechanical interference between engagement feature 351b and the lateral sides of engagement feature 331b (such side to side movement would be in and out of the plane of FIG. 22).

FIG. 22 shows that the tightening of set screw 380 within threads 335g results in clamping of retainer 350 onto the top surface of head 330 by the action of the lip 382 of the set screw pressing against an edge of the aperture in retainer 350. Retainer 350 includes an open channel 356 that extends from a midsection of retainer 350 to an edge of retainer 350, as best seen in FIG. 22. As the set screw is tightened, lip 382 compresses against corner 356a as the lip and corner are pulled toward each other by the screw. After tightening of set screw 380, the engagement features 351b and 331b remain captured to each other, with the mating of retainer 350 and the body 330a of the head bringing together the upper and lower rod paths, respectively, and creating an enclosed rod pathway 324 that is smooth and continuous, and free of surface interruptions.

Statements about various embodiments described herein include the following: (A) an implantable anchor assembly for support of a spinal rod, comprising a bone connector; further comprising a head having first and second opposing arms and forming a lower rod pathway therebetween, each said first and second opposing arm having the same one of a notch or a projection, the one of a notch or projection of said first arm facing one of a notch or projection of said second arm; further comprising a rod retainer having third and fourth opposing arms and forming an upper rod pathway therebetween, each said third and fourth opposing arm having the other of a notch or a projection, said third and fourth opposing arms being slidable to a position between said first and second opposing arms, said rod retainer having a threaded aperture; and further comprising and a set screw threadably receivable within the threaded aperture, wherein sliding said rod retainer toward the position elastically bends inward toward each other said third arm and said fourth arm, and said third and fourth arms each elastically bend outwards upon reaching the position, wherein at the position each one of the notch or projection of said first or second arms is received by a corresponding other of the notch or projection of said third or fourth arms, and tightening of said set screw within the aperture discourages inward bending of each projection with corresponding notch; (B) an implantable anchor assembly for support of a spinal rod, comprising a bone connector having an end adapted and configured for polyaxial support; further comprising a head supported by said bone connector and having first and second opposing arms and forming a first concave rod pathway therebetween, said first arm including a first engagement feature, said second opposing arm including a second engagement feature, each said opposing arm having a top surface, the top surface of said first arm including a first interlocking feature, the top surface of said second arm including a second interlocking feature; further comprising a rod retainer having third and fourth opposing arms and forming a second rod pathway therebetween, said third arm including a third engagement feature, said fourth opposing arm including a fourth engagement feature, said third and fourth opposing arms being slidable to a position between said first and second opposing arms; and further comprising a cap having two opposing sides, with the first opposing side including a third interlocking feature, the second opposing side including a fourth interlocking feature, wherein sliding said rod retainer toward the position interlocks the first interlocking feature with the third interlocking feature and interlocks the second interlocking feature with the fourth interlocking feature, wherein at the position the first engagement feature engages with the third engagement feature and the second engagement feature engages with the fourth engagement feature, wherein the engagements prevent sliding separation of said rod retainer from said head, and wherein the interlockings are adapted and configured to prevent the first and second opposing arms from separating; (C) an implantable anchor assembly for support of a spinal rod, comprising a bone connector having an end adapted and configured for polyaxial support, the end including a tool coupling feature for applying a torque to said bone connector, said bone connector having a first centerline; further comprising a head having first and second opposing arms and forming a lower rod pathway therebetween, each of said first and second arms including facing portions that are threaded, the threaded portions having a second centerline, the lower rod pathway having a smooth and continuous surface adapted and configured for sliding contact with the rod, said head including a pocket for receiving the end of said bone connector, said head including a through passageway that opens to the pocket, the through passageway having a third centerline that is non-parallel to the first centerline; further comprising a rod retainer having first and second end walls forming a channel therebetween, said rod retainer including an upper rod pathway, the upper pathway having a smooth and continuous surface adapted and configured for sliding contact with the rod; and further comprising a set screw located in the channel and threadably receivable by the threaded portions of said first and second arms, wherein said bone connector can be rotated relative to said head from a first position in which the first centerline is coaxial with the third centerline to a second position in which the first centerline is parallel to the second centerline; (D) an implantable anchor assembly for support of a spinal rod, comprising a bone connector having an end adapted and configured for polyaxial support, the end including a corridor adapted and configured for applying a torque to said bone connector; further comprising a head having first and second opposing arms and forming a lower rod pathway therebetween, each of said first and second arms including facing portions that are threaded, the lower pathway having a smooth and continuous surface adapted and configured for sliding contact with the rod, said head including a pocket for receiving the end of said bone connector, said head including a through passageway that opens to the pocket; further comprising a rod retainer having a body forming an upper rod pathway, the upper pathway having a smooth and continuous surface adapted and configured for sliding contact with the rod; further comprising a set screw threadably receivable by the threaded portions of said first and second arms; and further comprising a readily releasable locking member, said locking member including a projection, wherein said head can be rotated relative to said head to a position in which the corridor of said bone connector aligns with the through passageway of said head, and the projection can be placed in the aligned corridor and in through passageway; and (E) an implantable anchor assembly for support of a spinal rod, comprising a bone connector having an end adapted and configured for polyaxial support, the end including a tool coupling feature for applying a torque to said bone connector, said bone connector having a first centerline; further comprising a head having first and second opposing arms and forming a lower rod pathway therebetween, the lower pathway having a smooth and continuous surface adapted and configured for sliding contact with the rod along a second centerline, said head including a pocket for receiving the end of said bone connector, said head including a threaded through passageway that opens to the pocket, the through passageway having a third centerline; further comprising a rod retainer having an upper rod pathway having a smooth and continuous surface adapted and configured for sliding contact with the rod; and further comprising a set screw threadably receivable by the threaded through passageway of said head, wherein said bone connector can be rotated relative to said head from a first position in which the first centerline is coaxial with the third centerline to a second position in which the first centerline and the third centerline form an acute angle.

Still further embodiments are realized by combining any of previous statements (A)-(E) above with one or more of the statements that follow: (1) wherein the rod has a maximum cross sectional distance and when said rod retainer is in the position the assembled upper and lower rod pathways have a minimum cross section distance greater than the maximum cross sectional distance; (2) wherein said bone connector has a rounded head, said head includes a pocket for receiving therein the rounded head, and said head can rotate in at least one direction relative to said bone connector; (3) wherein said rod retainer includes a flexible portion between said third and fourth arms, and the tightening of said set screw within the threaded aperture increases the stiffness of the flexible portion; (4) wherein the lower rod pathway and the lower rod pathway coact to create a unified rod pathway adapted and configured for sliding contact with the rod when said rod retainer is placed at the position; (5) wherein the first interlocking feature and the second interlocking feature are each shaped as female dovetails and the third interlocking feature and the second interlocking feature are each shaped as male dovetails; (6) wherein the first interlocking feature and the second interlocking feature are each shaped as male dovetails and the third interlocking feature and the second interlocking feature are each shaped as female dovetails; (7) which further comprises a set screw, wherein said rod retainer includes a threaded aperture for threadably receiving said set screw, said cap includes a hole for passage therethrough of a portion of said set screw; (8) wherein said set screw passes through the hole before threadably engaging said rod retainer, and tightening of said set screw compresses at least a portion of said cap between said set screw and said rod retainer; (9) wherein the through passageway of said head does not intersect the pocket of said head; (10) wherein the upper pathway and the lower pathway each include a cross sectional shape that is partly circular; (11) wherein the lower pathway is unbroken by any aperture; (12) wherein the lower rod pathway and the lower rod pathway coact to create a unified rod pathway adapted and configured for sliding contact with the rod when said set screw is tight in the threaded portions of the first and second opposing arms; (13) wherein the lower pathway of said head does not provide access to the pocket of said head; (14) wherein said locking member includes a holding portion on an end of the projection, said holding portion having a cross sectional shape adapted and configured to prevent insertion of the holding portion into the through passageway; (15) wherein the projection of said locking member is adapted and configure to transmit to the end of said bone connector a torque applied to said head; (16) wherein said head includes a first interlocking feature, said rod retainer includes a second interlocking feature, and said first interlocking feature interlocks with the second interlocking feature by sliding of said rod retainer along the top of said head in a direction perpendicular to the second centerline; (17) wherein said head includes a first interlocking feature, said rod retainer includes a second interlocking feature, at least one of said first interlocking feature or the second interlocking feature include an elastically bendable portion, and said first interlocking feature interlocks with the second interlocking feature by releasing the elastically bent portion into the other of the first interlocking feature or the second interlocking feature; (18) wherein said head is a first head, and said set screw includes a screw head having a diameter larger than the major diameter of the set screw threads, and tightening of said set screw into threaded through passageway of said first head results in compression of said rod retainer by the screw head.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

What is claimed is:

1. An implantable anchor assembly for support of a spinal rod, comprising:
a bone connector;
a head having first and second opposing arms and forming a lower rod pathway therebetween, each said first and second opposing arm having the same one of a notch or a projection, the one of a notch or projection of said first arm facing one of a notch or projection of said second arm;
a rod retainer having third and fourth opposing arms and forming an upper rod pathway therebetween, each said third and fourth opposing arm having the other of a notch or a projection, said third and fourth opposing arms being slidable to a position between said first and second opposing arms, said rod retainer having a threaded aperture; and
a set screw threadably receivable within the threaded aperture,
wherein sliding said rod retainer toward the position elastically bends inward toward each other said third arm and said fourth arm,
wherein said third and fourth arms each elastically bend outwards upon reaching the position,
wherein at the position each one of the notch or projection of said first or second arms is received by a corresponding other of the notch or projection of said third or fourth arms, and
wherein tightening of said set screw within the aperture discourages inward bending of each projection with corresponding notch.

2. An implantable anchor assembly for support of a spinal rod, comprising:
a bone connector having an end adapted and configured for polyaxial support;
a head supported by said bone connector and having first and second opposing arms and forming a first concave rod pathway therebetween, said first arm including a first engagement feature, said second opposing arm including a second engagement feature, each said opposing arm having a top surface, the top surface of said first arm including a first interlocking feature, the top surface of said second arm including a second interlocking feature;
a rod retainer having third and fourth opposing arms and forming a second rod pathway therebetween, said third arm including a third engagement feature, said fourth opposing arm including a fourth engagement feature, said third and fourth opposing arms being slidable to a position between said first and second opposing arms; and
a cap having two opposing sides, with the first opposing side including a third interlocking feature, the second opposing side including a fourth interlocking feature,
wherein sliding said rod retainer toward the position interlocks the first interlocking feature with the third interlocking feature and interlocks the second interlocking feature with the fourth interlocking feature,
wherein at the position the first engagement feature engages with the third engagement feature and the second engagement feature engages with the fourth engagement feature,
wherein the engagements prevent sliding separation of said rod retainer from said head, and
wherein the interlockings are adapted and configured to prevent the first and second opposing arms from separating.

3. An implantable anchor assembly for support of a spinal rod, comprising:
a bone connector having an end adapted and configured for polyaxial support, the end including a tool coupling feature for applying a torque to said bone connector, said bone connector having a first centerline;
a head having first and second opposing arms and forming a lower rod pathway therebetween, each of said first and second arms including facing portions that are threaded, the threaded portions having a second centerline, the lower rod pathway having a smooth and continuous surface adapted and configured for sliding contact with the rod, said head including a pocket for receiving the end of said bone connector, said head including a through passageway that opens to the pocket, the through passageway having a third centerline that is non-parallel to the first centerline;
a rod retainer having first and second end walls forming a channel therebetween, said rod retainer including an upper rod pathway, the upper pathway having a smooth and continuous surface adapted and configured for sliding contact with the rod; and
a set screw located in the channel and threadably receivable by the threaded portions of said first and second arms,
wherein said bone connector can be rotated relative to said head from a first position in which the first centerline is coaxial with the third centerline to a second position in which the first centerline is parallel to the second centerline.

4. An implantable anchor assembly for support of a spinal rod, comprising:
a bone connector having an end adapted and configured for polyaxial support, the end including a corridor adapted and configured for applying a torque to said bone connector;
a head having first and second opposing arms and forming a lower rod pathway therebetween, each of said first and second arms including facing portions that are threaded, the lower pathway having a smooth and continuous surface adapted and configured for sliding contact with the rod, said head including a pocket for receiving the end of said bone connector, said head including a through passageway that opens to the pocket;
a rod retainer having a body forming an upper rod pathway, the upper pathway having a smooth and continuous surface adapted and configured for sliding contact with the rod;
a set screw threadably receivable by the threaded portions of said first and second arms; and
a readily releasable locking member, said locking member including a projection,
wherein said head can be rotated relative to said head to a position in which the corridor of said bone connector aligns with the through passageway of said head, and
wherein the projection can be placed in the aligned corridor and in through passageway.

5. An implantable anchor assembly for support of a spinal rod, comprising:
a bone connector having an end adapted and configured for polyaxial support, the end including a tool coupling feature for applying a torque to said bone connector, said bone connector having a first centerline;
a head having first and second opposing arms and forming a lower rod pathway therebetween, the lower pathway having a smooth and continuous surface adapted and configured for sliding contact with the rod along a second centerline, said head including a pocket for receiving the end of said bone connector, said head including a threaded through passageway that opens to the pocket, the through passageway having a third centerline;
a rod retainer having an upper rod pathway having a smooth and continuous surface adapted and configured for sliding contact with the rod; and
a set screw threadably receivable by the threaded through passageway of said head,
wherein said bone connector can be rotated relative to said head from a first position in which the first centerline is coaxial with the third centerline to a second position in which the first centerline and the third centerline form an acute angle.
